(19) United States
(12) Patent Application Publication
(10) Pub. No.: US 2025/0154090 A1
(43) Pub. Date: May 15, 2025

(54) PROCESS FOR PREPARING GAMMA,DELTA-UNSATURATED ALDEHYDES DERIVATIVES

(71) Applicant: Firmenich SA, Satigny (CH)

(72) Inventors: Oliver KNOPFF, Satigny (CH); Jean-Jacques RIEDHAUSER, Satigny (CH); Nicolas POIRIER, Satigny (CH); Luigi MARINONI, Satigny (CH)

(21) Appl. No.: 18/840,053

(22) PCT Filed: Mar. 1, 2023
(86) PCT No.: PCT/EP23/55069

§ 371 Date: Aug. 20, 2024

(30) Foreign Application Priority Data
Mar. 1, 2022 (EP) 22159463.3

(51) Int. Cl. C07C 45/50; B01J 31/18; B01J 31/20; B01J 31/22; C07C 45/67; C07C 69/78

(52) U.S. Cl. CPC C07C 45/50; B01J 31/185; B01J 31/20; B01J 31/2234; C07C 45/676; C07C 69/78; B01J 2231/321; B01J 2531/004; B01J 2531/822

(57) ABSTRACT

Described herein is a process for preparing a compound of formula (I). A compound of formula (III), a compound of formula (IV) and a compound of formula (V) are also described herein.

### TECHNICAL FIELD

The present invention relates to the field of organic synthesis and more specifically it concerns a process for preparing compound of formula (I). The compound of formula (III), the compound of formula (IV) and the compound of formula (V) are also part of the invention.

### BACKGROUND OF THE INVENTION

In the perfumery industry, there is a constant need to provide compounds imparting novel organoleptic notes. In particular, there is an interest towards aldehydic notes which represent one of the key organoleptic facets of the lily of the valley odor. So, compounds imparting said note are particularly sought after to reconstitute the delicate floral odor of muguet which does not survive even the mildest of extraction methods to yield an essential oil. Gamma,delta-unsaturated aldehydes of formula (I) represent compounds imparting note of the muguet-aldehydic olfactive family, such as, for example, compounds reported in WO2010052635, WO2013117433 or WO2015000821. However, the access to these compounds are tedious providing the desired compounds with low yield and/or selectivity.

Being products of industrial interest, there is always a need for new processes showing an improved selectivity and yield or productivity.

The compounds of formula (III), (IV) and (V) which are an object of the present invention, have never been reported or suggested in the context of the preparation of compounds of formula (I).

### SUMMARY OF THE INVENTION

The invention relates to a novel process allowing the preparation of compound of formula (I) with a high yield and high selectivity starting from compound of formula (II). The invention process represents a new efficient route toward compound of formula (I).

So, the first object of the present invention is a process for the preparation of a compound of formula

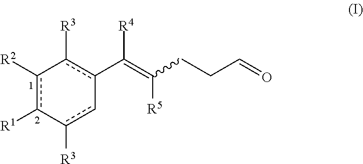

in the form of any one of its stereoisomers or a mixture thereof, and wherein all the dotted lines represent double bonds or the dotted line between carbon 1 and carbon 2 is a carbon-carbon single bond or a carbon-carbon double bond and the other dotted lines are carbon-carbon single bonds; each R¹, R² and R³, independently from each other, represent a hydrogen atom, a C₁₋₃ alkoxy group, a C₁₋₆ alkyl group or a C₂₋₆ alkenyl group, each optionally substituted by a hydroxy or C₁₋₃ alkoxy group; or R¹ and R², are taken together and form a C₃₋₈ cycloalkyl or C₅₋₈ cycloalkenyl group; or both R³, taken together, are a C₁₋₃ alkanediyl; R⁴ and R⁵, independently from each other, are a hydrogen atom, a methyl or an ethyl group;

comprising a hydroformylation and an elimination step starting from compound of formula (II)

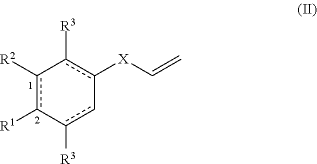

in the form of any one of its stereoisomers or a mixture thereof, and wherein the dotted lines, R¹, R² and R³ have the same meaning as defined for compound of formula (I) and X represents a CR⁴(OC(═O)R⁶)CHR⁵ or CHR⁴C(OC(═O)R⁶)(R⁵) group wherein R⁴ and R⁵ have the same meaning as defined for compound of formula (I) and R⁶ is a hydrogen atom, a C₁₋₃ alkoxy group, a phenyl group or C₁₋₃ alkyl group optionally substituted by one to three halogen atoms.

A second object of the present invention is a compound of formula

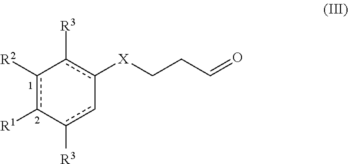

in the form of any one of its stereoisomers or a mixture thereof, and wherein all the dotted lines represent double bonds or the dotted line between carbon 1 and carbon 2 is a carbon-carbon single bond or a carbon-carbon double bond and the other dotted lines are carbon-carbon single bonds; each R¹, R² and R³, independently from each other, represent a hydrogen atom, a C₁₋₃ alkoxy group, a C₁₋₆ alkyl group or a C₂₋₆ alkenyl group, each optionally substituted by a hydroxy or C₁₋₃ alkoxy group; or R¹ and R², are taken together and form a C₃₋₈ cycloalkyl or C₅₋₈ cycloalkenyl group; or both R³, taken together, are a C₁₋₃ alkanediyl group; X represents a CR⁴(OC(═O)R⁶)CHR⁵ or CHR⁴C(OC(═O)R⁶)(R⁵) group wherein R⁴ and R⁵, independently from each other, are a hydrogen atom, a methyl or an ethyl group and R⁶ is a hydrogen atom, a C₁₋₃ alkoxy group, a phenyl group or C₁₋₃ alkyl group optionally substituted by one to three halogen atoms; provided that 5-oxo-1-phenylpentyl acetate is excluded.

Another object of the present invention is compound of formula

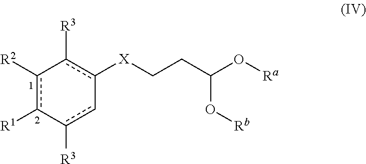

in the form of any one of its stereoisomers or a mixture thereof, and wherein all the dotted lines represent double bonds or the dotted line between carbon 1 and carbon 2 is a carbon-carbon single bond or a carbon-carbon double bond and the other dotted lines are carbon-carbon single bonds; each R¹, R² and R³, independently from each other, represent a hydrogen atom, a C₁₋₃ alkoxy group, a C₁₋₆ alkyl group or a C₂₋₆ alkenyl group, each optionally substituted by a hydroxy or C₁₋₃ alkoxy group; or R¹ and R², are taken together and form a C₃₋₈ cycloalkyl or C₅₋₈ cycloalkenyl group; or both R³, taken together, are a C₁₋₃ alkanediyl group; X represents a CR⁴(OC(═O)R⁶)CHR⁵ or CHR⁴C(OC(═O)R⁶)(R⁵) group wherein R⁴ and R⁵, independently from each other, are a hydrogen atom, a methyl or an ethyl group and R⁶ is a hydrogen atom, a C₁₋₃ alkoxy group, a phenyl group or C₁₋₃ alkyl group optionally substituted by one to three halogen atoms; R^{a} and R^{b}, independently from each other, represent a C₁₋₄ alkyl group or R^{a} and R^{b} are taken together and represent a C₂₋₆ alkanediyl group, preferably R^{a} and R^{b} are taken together and represent a (CH₂)ₙ group wherein n is 2 or 3.

A further object of the present invention is a compound of formula

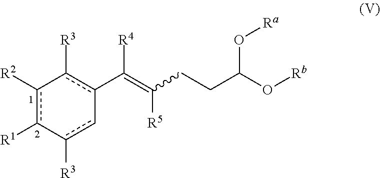

in the form of any one of its stereoisomers or a mixture thereof, and wherein all the dotted lines represent double bonds or the dotted line between carbon 1 and carbon 2 is a carbon-carbon single bond or a carbon-carbon double bond and the other dotted lines are carbon-carbon single bonds; each R¹, R² and R³, independently from each other, represent a hydrogen atom, a C₁₋₃ alkoxy group, a C₁₋₆ alkyl group or a C₂₋₆ alkenyl group, each optionally substituted by a hydroxy or C₁₋₃ alkoxy group; or R¹ and R², are taken together and form a C₃₋₈ cycloalkyl or C₅₋₈ cycloalkenyl group; or both R³, taken together, are a C₁₋₃ alkanediyl group; R⁴ and R⁵, independently from each other, are a hydrogen atom, a methyl or an ethyl group; R^{a} and R^{b}, independently from each other, represent a C₁₋₄ alkyl group or R^{a} and R^{b} are taken together and represent a C₂₋₆ alkanediyl group, preferably R^{a} and R^{b} are taken together and represent a (CH₂) n group wherein n is 2 or 3; provided that 2-(4-cyclohexylbut-3-en-1-yl)-1,3-dioxolane, 2-(4-phenylbut-3-en-1-yl)-1,3-dioxolane and 2-(4-phenylpent-3-en-1-yl)-1,3-dioxolane are excluded.

### DESCRIPTION OF THE INVENTION

It has now been surprisingly found that valuable compound of formula (I) can be obtained from new chemical intermediates, as defined herein below in formula (III), (IV) and (V). The invention's process represents a new route toward compounds of formula (I) with overall higher yield, compared to the methods known from the prior art.

So, the first object of the invention is a process for the preparation of a compound of formula

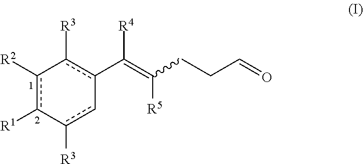

in the form of any one of its stereoisomers or a mixture thereof, and wherein all the dotted lines represent double bonds or the dotted line between carbon 1 and carbon 2 is a carbon-carbon single bond or a carbon-carbon double bond and the other dotted lines are carbon-carbon single bonds; each R¹, R² and R³, independently from each other, represent a hydrogen atom, a C₁₋₃ alkoxy group, a C₁₋₆ alkyl group or a C₂₋₆ alkenyl group, each optionally substituted by a hydroxy or C₁₋₃ alkoxy group; or R¹ and R², are taken together and form a C₃₋₈ cycloalkyl or C₅₋₈ cycloalkenyl group; or both R³, taken together, are a C₁₋₃ alkanediyl group; R⁴ and R⁵, independently from each other, are a hydrogen atom, a methyl or an ethyl group;

comprising a hydroformylation and an elimination step starting from compound of formula (II)

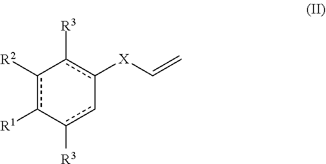

in the form of any one of its stereoisomers or a mixture thereof, and wherein the dotted lines, R¹, R² and R³ have the same meaning as defined for compound of formula (I) and X represents a —C(R⁴)(OC(═O)R⁶)—CH(R⁵)— or —CH(R⁴)—C(OC(═O)R⁶)(R⁵)— group wherein R⁴ and R⁵ have the same meaning as defined for compound of formula (I) and R⁶ is a hydrogen atom, a C₁₋₃ alkoxy group, a phenyl group or C₁₋₃ alkyl group optionally substituted by one to three halogen atoms.

For the sake of clarity, by the expression “any one of its stereoisomers or a mixture thereof”, or the similar, it is meant the normal meaning understood by a person skilled in the art, i.e. that the compound of formula (I) and (II) can be a pure enantiomer or a mixture of enantiomers. In other words, the compound of formula (I) and (II) may possess at least one stereocenter which can have two different stereochemistries (e.g. R or S). The compounds of formula (I) and (II) may even be in the form of a pure enantiomer or in the form of a mixture of enantiomers. The compounds of formula (I) and (II) may even be in the form of a pure diastereoisomer or in the form of a mixture of diastereoisomers when compounds of formula (I) and (II) possess more than one stereocenter. The compounds of formula (I) and (II) can be in a racemic form or scalemic form. Therefore, the compounds of formula (I) and (II) can be one stereoisomer or in the form of a composition of matter comprising, or consisting of, various stereoisomers.

For the sake of clarity, by the wavy bond in compound of formula (I), or the similar, it is meant the normal meaning understood by a person skilled in the art, i.e. that the double bond may have a cis configuration corresponding to the Z isomer, a trans configuration corresponding to the E isomer or a mixture thereof. In other words, the compound of formula (I) may be in the form of its E or Z isomer or of a mixture thereof, e.g. the invention process leads to a composition of matter consisting of one or more compounds of formula (I), having the same chemical structure but differing by the configuration of the double bond. In particular, compound (I) can be in the form of a mixture consisting of isomers E and Z and wherein said isomer E represents at least 25% of the total mixture, at least 35%, at least 50%, or even at least 75% (i.e a mixture E/Z comprised between 75/25 and 100/0).

For the sake of clarity, by the expression “all the dotted lines represent double bonds or the dotted line between carbon 1 and carbon 2 is a carbon-carbon single bond or a carbon-carbon double bond and the other dotted lines are carbon-carbon single bonds”, or the similar, it is meant the normal meaning understood by a person skilled in the art, i.e. that the whole bonding (solid and dotted line) between the carbon atoms connected by said dotted line, e.g. carbon 1 and 2, is a carbon-carbon single or double bond.

The term “optionally” is understood that a certain group to be optionally substituted can or cannot be substituted with a certain functional group.

For the sake of clarity, by the expression “comprising a hydroformylation and an elimination step”, it is meant that the hydroformylation reaction and the elimination reaction may be performed in any order. In other words, the invention process may comprise a hydroformylation step followed by an elimination step or the invention process may comprise an elimination step followed by a hydroformylation step.

The terms “alkyl” and “alkenyl” are understood as comprising branched and linear alkyl and alkenyl groups. The terms “alkenyl” and “cycloalkenyl” are understood as comprising 1, 2 or 3 olefinic double bonds, preferably 1 or 2 olefinic double bonds. The terms “cycloalkyl” and “cycloalkenyl” are understood as comprising a monocyclic or fused, spiro and/or bridged bicyclic or tricyclic cycloalkyl and cycloalkenyl, groups, preferably monocyclic cycloalkyl and cycloalkenyl groups.

For the sake of clarity, by the expression “R¹ and R², are taken together and form a C₃₋₈ cycloalkyl or C₅₋₈ cycloalkenyl group”, it is meant that the carbon atoms to which both groups are bonded are included into the C₅₋₈ cycloalkyl or C₅₋₈ cycloalkenyl group.

The term “alkanediyl” is understood as comprising branched and linear alkanediyl group.

According to any embodiment of the invention, all dotted lines may be double bonds. In other words, the compound of formula (I) is of formula

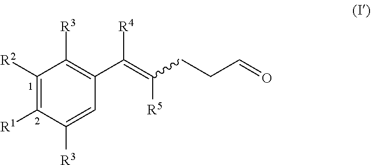

in the form of any one of its stereoisomers or a mixture thereof, and wherein R¹, R², R³, R⁴ and R⁵ have the same meaning as defined above; and
the compound of formula (II) is of formula

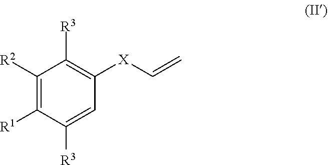

in the form of any one of its stereoisomers or a mixture thereof, and wherein R¹, R², R³ and X have the same meaning as defined above.

According to any embodiment of the invention, R⁴ may be a hydrogen atom or a methyl group. Even more particularly, R⁴ may be a hydrogen atom.

According to any embodiment of the invention, R⁵ may be a methyl or an ethyl group. Even more particularly, R⁵ may be a methyl group.

According to any embodiment of the invention, R³ may be, independently from each other, a hydrogen atom, a methoxy group, an ethoxy group, a C₁₋₄ alkyl group or a C₂₋₄ alkenyl group, each optionally substituted by a hydroxy, methoxy or ethoxy group. Particularly, R³ may be, independently from each other, a hydrogen atom, a C₁₋₃ alkyl group or a C₂₋₃ alkenyl group, each optionally substituted by a hydroxy or methoxy group. Particularly, R³ may be, independently from each other, a hydrogen atom or a C₁₋₃ alkyl group. Particularly, R³ may be, independently from each other, a hydrogen atom or a methyl or ethyl group. Even more particularly, R³ may be a hydrogen atom.

According to any embodiment of the invention, when the dotted line between carbon 1 and carbon 2 is a carbon-carbon single bond or a carbon-carbon double bond and the other dotted lines are carbon-carbon single bonds, then both R³, taken together, may be a C₁₋₃ alkanediyl group. Particularly, both R³, taken together, may be a CH₂, a CHMe or a C(Me)₂ group. Even more particularly, R³, taken together, may be a CH₂ group.

According to any embodiment of the invention, the compound of formula (I) is of formula

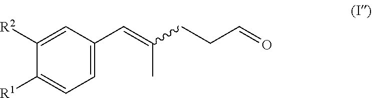

in the form of any one of its stereoisomers or a mixture thereof, and wherein each R¹ and R² have the same meaning as defined above;
and said compound of formula (II) is of formula

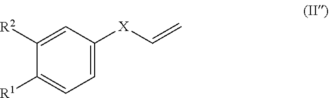

in the form of any one of its stereoisomers or a mixture thereof, and wherein each R¹ and R² have the same meaning as defined above and X may be a CH(OC(═O)R⁶)CHMe or CH₂C(OC(═O)R⁶)(Me) wherein R⁶ is a C₁₋₃ alkoxy group, a phenyl group or C₁₋₃ alkyl group optionally substituted by one to three halogen atoms.

According to any embodiment of the invention, R⁶ may be a hydrogen atom, a phenyl group or a C₁₋₃ alkyl group optionally substituted by one to three halogen atoms. Particularly, R⁶ may be a hydrogen atom or a C₁₋₃ alkyl group optionally substituted by one to three fluorine atoms. Particularly, R⁶ may be a methyl, trifluoromethyl or an ethyl group. Even more particularly, R⁶ may be a methyl group.

According to any embodiment of the invention, X may be a CHR⁴C(OC(═O) R⁶) (R⁵) group wherein R⁴, R⁵ and R⁶ have the same meaning as defined above.

According to any embodiment of the invention, R¹ may be, independently from each other, a hydrogen atom, a methoxy group, an ethoxy group, a C₁₋₄ alkyl group or a C₂₋₄ alkenyl group, each optionally substituted by a hydroxy, methoxy or ethoxy group. Particularly, R¹ may be, independently from each other, a hydrogen atom, a C₁₋₃ alkyl group or a C₂₋₃ alkenyl group, each optionally substituted by a hydroxy or methoxy group. Particularly, R¹ may be, independently from each other, a hydrogen atom or a methyl or ethyl group. Even more particularly, R¹ may be a methyl group.

According to any embodiment of the invention, R² may be, independently from each other, a hydrogen atom, a methoxy group, an ethoxy group, a C₁₋₄ alkyl group or a C₂₋₄ alkenyl group, each optionally substituted by a hydroxy, methoxy or ethoxy group. Particularly, R² may be, independently from each other, a hydrogen atom, a C₁₋₃ alkyl group or a C₂₋₃ alkenyl group, each optionally substituted by a hydroxy or methoxy group. Particularly, R² may be, independently from each other, a hydrogen atom or a methyl or ethyl group. Even more particularly, R² may be a hydrogen atom.

According to any embodiment of the invention, the compound of formula (II) may be prepared according to method known in the state of the art.

According to a particular embodiment of the invention, the invention's process comprises a hydroformylation followed by an elimination step starting from compound of formula (II). The hydroformylation of compound of formula (II) provides a compound of formula

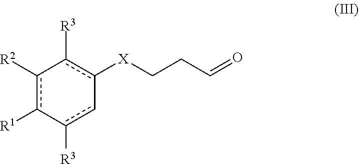

in the form of any one of its stereoisomers or a mixture thereof, and wherein the dotted lines, X, R¹, R² and, R³ have the same meaning as defined above.

For the sake of clarity, by the expression “hydroformylation”, or the similar, it is meant the normal meaning understood by a person skilled in the art, i.e. the reaction is performed in a presence of a metal catalyst such as Rhodium, Cobalt or Platinum complex, preferably a Rhodium complex, carbon monoxide, hydrogen and optionally a phosphorous containing ligand.

According to any embodiment of the invention, the hydroformylation is performed in a presence of a Rhodium complex. The Rhodium complexes that can be used in the present invention include but are not limited to Rh(acac)(CO)₂, RhCl₃, Rh₂AcO₄, [Rh(OAc)(COD)]₂, Rh₄(CO)₁₂, Rh₆(CO)₁₆, RhCl(CO)(PPh₃)₂, Rh(C₂H₄)₂(acac), [Rh(Cl)(COD)]₂, [Rh(Cl)(COE)₂]₂, [Rh(OAc)(CO)₂]₂, Rh(acac)(COD), HRh(CO)(PPh₃)₃, RhCl(PPh₃)₃, [Rh(NBD)₂]BF₄, [Rh(OMe)(COD)]₂ and [Rh(OH)(COD)]₂ wherein acac represents an acetyl acetonate group, Ac an acetyl group, COD a 1,5-cyclooctadiene group, COE a cyclooctene group, Ph a phenyl group. Particularly, the Rhodium complex may be selected from the group consisting of Rh(acac)(CO)₂, [Rh(OAc)(COD)]₂, RhCl(CO)(PPh₃)₂, Rh(C₂H₄)₂(acac), [Rh(CI)(COD)]₂, [Rh(CI)(COE)₂]₂, [Rh(OAc)(CO)₂]₂, Rh(acac)(COD), HRh(CO)(PPh₃)₃, RhCl(PPh₃)₃, [Rh(NBD)₂]BF₄, [Rh(OMe)(COD)]₂, and [Rh(OH)(COD)]₂. Even more particularly, the Rhodium complex may be selected from the group consisting of Rh(acac)(CO)₂, Rh(acac)(COD), HRh(CO)(PPh₃)₃, [Rh(OMe)(COD)]₂ and [Rh(OH)(COD)]₂. Said complex can be added into the reaction medium of the invention's process in a large range of concentrations. As non-limiting examples, one can cite as complex concentration values those ranging from about 0.0005 mol % to about 5 mol %, relative to the amount of substrate, preferably from 0.001 mol % to about 5 mol %, relative to the amount of substrate. Preferably, the complex concentration will be comprised between 0.0025 mol % to 2 mol %. It goes without saying that the optimum concentration of the complex will depend, as the person skilled in the art knows, on the nature of the latter, on the nature of the substrate, on the nature of the ligand, on the reaction temperature as well as on the desired time of reaction.

According to any embodiment of the invention, the hydroformylation is performed in a presence of a mono- or bidentate phosphorous ligand. Particularly, the phosphorous ligand may be a bidentate phosphorous ligand.

According to any embodiment of the present invention, the hydroformylation may be performed in a presence of a monodentate phosphorous ligand of formula PR⁸₃, wherein R⁸ is a C₁-C₁₂ group, such as linear, branched or cyclic alkyl, alkoxy or aryloxy group optionally substituted, substituted or unsubstituted phenyl, diphenyl, 2-furanyl, naphthyl or di-naphthyl group, or two R⁸ groups are taken together and form a phosphatrioxa-adamantane and the other R⁸ group has the same meaning as above. More particularly R⁸ may represent a substituted or unsubstituted phenyl, diphenyl, naphthyl or di-naphthyl group. Possible substituents are those cited below for the group R⁹. Preferably, the monodentate phosphorous ligand is a triphenylphosphine.

According to any one of the above embodiments, the hydroformylation may be performed in presence of a bidentate phosphorous ligand of formula

(R⁹)₂P-Q-P(R⁹)₂ (A)
wherein each R⁹, taken separately, represents C₄ heteroaryl group, a C₆₋₁₀ aromatic group optionally substituted or a cyclohexyl group optionally substituted, or the two R⁹ bonded to the same P atom, taken together, represent a 1,1′-biphenyl-2,2′-dioxy, a 1,1′-biphenyl-2,2′-dimetyl or a 1,1′-biphenyl]-2,2′-diyl, each optionally substituted; and

Q represents a group of formula
a)

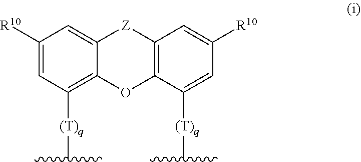

wherein q is 0 or 1, each T, independently from each other, represent an oxygen atom or a CH₂ group, each R¹⁰, independently from each other, represents a hydrogen atom or a C₁₋₈ alkyl group, and Z represents an oxygen or sulfur atom or a C(R¹¹)₂, Si(R¹²)₂ or NR11 group, in which R¹¹ is a hydrogen atom or a R¹² group, R¹² representing a C₁₋₄ linear or branched alkyl group, preferably methyl group; or
b)

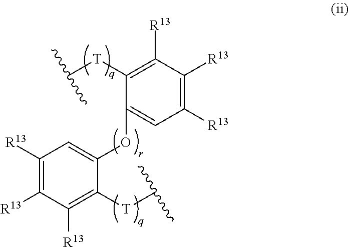

in the form of any one of its enantiomers, and wherein q is 0 or 1, r is 0 or 1, each T, independently from each other, represent an oxygen atom or a CH₂ group, R¹³, independently from each other, represent a hydrogen atom, a methoxy group or a C₁-4 alkyl optionally substituted by one to three halogen atoms or alkoxy groups; or two adjacent R¹³ may be taken together and represent a (CH) 4 group;
c)

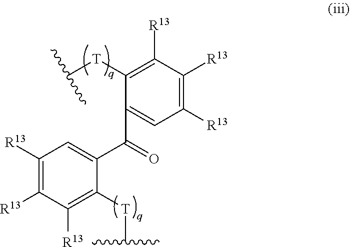

in the form of any one of its enantiomers, and wherein T, q and R¹³ have the same meaning as above;
   and the wavy lines indicate the position of the bond between said Q group and the rest of the compound (A).

According to any one of the above embodiments, Q may be a group of formula (i) or (ii).

According to any one of the above embodiments, each R⁹ may be a furan-2-yl group, a 1H-pyrrol-1-yl group, a C₆₋₁₀ aromatic group optionally substituted or a cyclohexyl group optionally substituted.

According to any one of the above embodiments, by “aromatic group or ring” it is meant a phenyl or naphthyl group, and in particular a phenyl group.

According to any one of the above embodiments, each R⁹ may be a phenyl group, a cyclohexyl group, a 3,5-dimethyl-phenyl, a 3,5-di(CF₃)-phenyl, a 3,5-dimethyl-4-methoxy-phenyl group.

According to any one of the above embodiments, the R¹⁰ may be a hydrogen atom.

According to any one of the above embodiments, Z may be a CMe₂, SiMe₂, NH or NMe group. Particularly, Z may be a CMe₂ group.

According to any one of the above embodiments, non-limiting examples of possible substituents of R⁹ are one, two, three or four groups selected amongst the halogen atoms, or C₁₋₁₀ alkoxy, alkyl, alkenyl, pyridyl or perhalo-hydrocarbon group. Two substituents may be taken together to form a C₄₋₈ cycloalkyl group. The expression “perhalo-hydrocarbon” has here the usual meaning in the art, e.g. a group such as CF₃ for instance. In particular said substituents are one or two halogen atoms, such as F or Cl, or C₁₋₄ alkoxy or alkyl groups, or CF₃ groups.

According to any one of the above embodiments, said R⁹ may be non-substituted.

According to any one of the above embodiments, the ligand of formula (A) can be in a racemic or optically active form.

Non limiting example of bidendate phosphorous ligand may include 2,2′-bis((di(1H-pyrrol-1-yl)phosphanyl)oxy)-1,1′-binaphthalene, 1,1′-((naphthalen-2-yloxy)phosphanediyl)bis(1H-pyrrole), 2,2′-bis((di(1H-pyrrol-1-yl) phosphanyl)oxy)-1,1′-biphenyl, (9,9-dimethyl-9H-xanthene-4,5-diyl)bis(diphenylphosphine), 2,2′-bis((di (1H-pyrrol-1-yl)phosphaneyl)oxy)-5,5′,6,6′,7,7′,8,8′-octahydro-1, l′-binaphthalene, 1,1′,1″,1″-(((2,7-di-tert-butyl-9,9-dimethyl-9H-xanthene-4,5-diyl)bis(oxy))bis(phosphanetriyl))tetrakis(1H-pyrrole), 6,6′-[(3,3′-Di-tert-butyl-5,5′-dimethoxy-1,1′-biphenyl-2,2′-diyl)bis(oxy)] bis(dibenzo[d,f] [1,3,2]dioxaphosphepin), (Oxydi-2,1-phenylene)bis(diphenylphosphine), 2,2′-Bis(diphenylphosphinomethyl)-1,1′-biphenyl, 4,6-bis(diphenylphosphanyl)-10H-phenoxazine, 2-((3,3′-di-tert-butyl-2′-((4,8-di-tert-butyl-2,10-dimethoxydibenzo[d,f] [1,3,2]dioxaphosphepin-6-yl)oxy)-5,5′-dimethoxy-[1,1′-biphenyl]-2-yl)oxy)-4H-naphtho[2,3-d][1,3,2]dioxaphosphinin-4-one, 2-((3,3′-di-tert-butyl-2′-((4,8-di-tert-butyl-2,10-dimethoxydibenzo[d,f] [1,3,2]dioxaphosphepin-6-yl)oxy)-5,5′-dimethoxy-[1,1′-biphenyl]-2-yl)oxy)-8-methyl-4H-benzo[d] [1,3,2]dioxaphosphinin-4-one, (15, 1'S)-(−)-(2,7-di-tert-butyl-9,9-dimethyl-9H-xanthene-4,5-diyl)bis((1-naphthyl) (phenyl)phosphine), (15,1'S)-(−)-(2,7-di-tert-butyl-9,9-dimethyl-9H-xanthene-4,5-diyl)bis((4-methylphenyl) (phenyl)phosphine), 8-methyl-2-((3,3′,5,5′-tetra-tert-butyl-2′-((2,4,8,10-tetra-ter-butyldibenzo[d,f] [1,3,2] dioxaphosphepin-6-yl)oxy)-[1,1′-biphenyl]-2-yl)oxy)-4H-benzo[ď] [1,3,2]dioxaphosphinin-4-one, 2-((3,3′-di-tert-butyl-2′-((4,8-di-tert-butyl-2,10-dimethoxydibenzo[d,f] [1,3,2]dioxaphosphepin-6-yl)oxy)-5,5′-dimethoxy-[1,1′-biphenyl]-2-yl)oxy)-8-isopropyl-5-methyl-4H-benzo[d] [1,3,2]dioxaphosphinin-4-one, (15,1'S′)-(+)-(9,9-Dimethyl-9H-xanthene-4,5-diyl)bis((2-methoxyphenyl) (phenyl)phosphine), (15,1'S)-(+)-(2,7-di-tert-butyl-9,9-dimethyl-9H-xanthene-4,5-diyl)bis((2-methoxyphenyl) (phenyl)phosphine), (15,1'S)-(+)-(9,9-Dimethyl-9H-xanthene-4,5-diyl)bis((2-methylphenyl) (phenyl)phosphine), (15, 1'S)-(−)-(9,9-Dimethyl-9H-xanthene-4,5-diyl)bis(naphthalen-2-yl(phenyl)phosphine), (15,1'S)-(−)-(9,9-Dimethyl-9H-xanthene-4,5-diyl)bis((4-methoxyphenyl) (phenyl)phosphine), (15,1'S)-(−)-(2,7-di-tert-butyl-9,9-dimethyl-9H-xanthene-4,5-diyl)bis((2-naphthyl) (phenyl)phosphine), (15,1'S)-(−)-(9,9-Dimethyl-9H-xanthene-4,5-diyl)bis(naphthalen-1-yl(phenyl)phosphine), (15,1'S)-(+)-(2,7-di-tert-butyl-9,9-dimethyl-9H-xanthene-4,5-diyl)bis((2-isopropoxyphenyl) (phenyl)phosphine), (15, 1'S)-(+)-(2,7-di-tert-butyl-9,9-dimethyl-9H-xanthene-4,5-diyl)bis((2-isopropylphenyl) (phenyl)phosphine) or (15,1'S)-(−)-(2,7-di-tert.-butyl-9,9-dimethyl-9H-xanthen-4,5-diyl)bis((dibenzo[b,d]-furan-4-yl) (phenyl)phosphine), (2,7-di-tert-butyl-9,9-dimethyl-9H-xanthene-4,5-diyl)bis((4-methoxyphenyl) (phenyl)phosphane), (4,4′,6,6′-Tetramethoxybiphenyl-2,2′-diyl)bis {bis [3,5-bis(trifluoromethyl)phenyl] phosphine}.

Particularly, the ligand is a bidentate phosporous ligand which may be selected from the group consisting of (9,9-dimethyl-9H-xanthene-4,5-diyl)bis(diphenylphosphine), 1,1′,1″,1′″-(((2,7-di-tert-butyl-9,9-dimethyl-9H-xanthene-4,5-diyl)bis(oxy))bis(phosphanetriyl))tetrakis(1H-pyrrole), 6,6′-[(3,3′-Di-tert-butyl-5,5′-dimethoxy-1, l′-biphenyl-2,2′-diyl)bis(oxy)] bis(dibenzo[d.f] [1,3,2]dioxaphosphepin), (Oxydi-2,1-phenylene)bis(diphenylphosphine), 2,2′-Bis(diphenylphosphinomethyl)-1,1′-biphenyl, 4,6-bis(diphenylphosphanyl)-10H-phenoxazine, 2-((3,3′-di-tert-butyl-2′-((4,8-di-tert-butyl-2,10-dimethoxydibenzo[d,f] [1,3,2]dioxaphosphepin-6-yl)oxy)-5,5′-dimethoxy-[1,1′-biphenyl]-2-yl)oxy)-4H-naphtho[2,3-d][1,3,2]dioxaphosphinin-4-one, 2-((3,3′-di-tert-butyl-2′-((4,8-di-tert-butyl-2,10-dimethoxydibenzo[d,f] [1,3,2]dioxaphosphepin-6-yl)oxy)-5,5′-dimethoxy-[1,1′-biphenyl]-2-yl)oxy)-8-methyl-4H-benzo[d] [1,3,2]dioxaphosphinin-4-one, (15,1'S)-(−)-(2,7-di-tert-butyl-9,9-dimethyl-9H-xanthene-4,5-diyl)bis((1-naphthyl) (phenyl)phosphine) or (15,1'S)-(−)-(2,7-di-tert-butyl-9,9-dimethyl-9H-xanthene-4,5-diyl)bis((4-methylphenyl) (phenyl)phosphine).

The phosphorous ligand can be added into the reaction medium of the invention's process in a large range of concentrations. As non-limiting examples, one can cite as phosphorous ligand concentration values those ranging from about 0.001 mol % to about 50 mol %, relative to the amount of the of substrate, preferably from 0.005 mol % to about 50 mol %, relative to the amount of the of substrate, preferably from about 0.005 mol % to about 15 mol %, relative to the amount of the of substrate. The optimum concentration of the phosphorous ligand will depend, as the person skilled in the art knows, on the nature 20) of the latter, on the nature of the substrate, on the nature of the metal complex, on the reaction temperature as well as on the desired time of reaction.

According to any one of the above embodiments, carbon monoxide and hydrogen gas may be generated in situ by known methods by the person skilled in the art, e.g. from methyl formate, formic acid, or formaldehyde. The CO/H₂ gas volume ratio is comprised between 2/1 to 1/5, preferably between 1/1 to 1/5 or preferably between 2/1 to 1/2, preferably between 1.5/1 to 1/1.5 and more preferably the ratio is 1/1.

The reaction can be carried out in the presence or absence of a solvent. When a solvent is required or used for practical reasons, then any solvent current in such reaction type can be used for the purposes of the invention. Non-limiting examples include C₆₋₁₂ aromatic solvents such as toluene, 1,3-diisopropylbenzene, cumene or pseudocumene, or mixtures thereof, alcoholic solvents such as methanol, ethanol, 2-methylbutan-2-ol or mixtures thereof, hydrocarbon solvents such as cyclohexane, heptane or mixtures thereof, esteric solvent such as n-butyl acetate, iso-propyl acetate, ethyl acetate or ethereal solvents such as methyl tetrahydrofuran, tetrahydrofuran or mixtures thereof. The choice of the solvent is function of the nature of the substrate and/or catalyst and the person skilled in the art is well able to select the solvent most suitable in each case to optimize the reaction.

The hydroformylation reaction can be carried out at a temperature in the range comprised between 50° C. and 150° C., more preferably in the range comprised between 80° C. and 130° C., or even between 90° C. and 110° C. Of course, a person skilled in the art is also able to select the preferred temperature according to the melting and boiling point of the starting and final products as well as the desired time of reaction or conversion.

The hydroformylation can be carried out at a CO/H₂ pressure comprised between 1 bar and 50 bar, preferably in the range of between 10 bar and 50 bar, more preferably in the range of between 10 bar and 25 bar. Of course, a person skilled in the art is well able to adjust the pressure as a function of the catalyst load and of the dilution of the substrate in the solvent.

According to any embodiment of the invention, the aldehyde group of compound of formula (III) may be protected before the elimination step or the elimination step may be performed directly on compound of formula (III) providing compound of formula (I). When the elimination step is performed on compound of formula (III), the elimination is performed under acidic conditions or under thermal pyrolysis, particularly under thermal pyrolysis. The acid may be selected from the group consisting of pTsOH, MsOH, TfOH, H₂SO₄, H₃PO₄, KHSO₄, NaHSO₄, oxalic acid, formic acid, BF₃ Et₂O, BF₃ AcOH, Alox acidic (Axsorb A2-5, Al₂O₃ 504C), Amberlyst 15, SiO₂, TFA, Wayphos, polyphosphoric acid, Zeolite (CBV 21A sold by Zeolyst, CBV 780 sold by Zeolyst, CP814E sold by Zeolyst), boric acid, Al₂(SO₄)₃, CSA, Pyridinium p-toluenesulfonate, ZnBr₂, K10-S300 (Bentonite) sold by Clariant, F24 X (Clay) sold by EP minerals, Siral® 40 HPV sold by Sasol, HCl, HBr, Zn(SO₄)₂, ZnCl₂ Mgl₂, and a mixture thereof. The thermal pyrolysis may be carried out at a temperature comprised in the range between 300° C. and 600° C.

The elimination reaction on the aldehydic substrate can be carried out in the presence or absence of a solvent. When a solvent is required or used for practical reasons, then any solvent current in such reaction type can be used for the purposes of the invention. Non-limiting examples include C₆₋₁₂ aromatic solvents such as toluene, xylene, 1,3-diisopropylbenzene, cumene or pseudocumene, or mixtures thereof, chlorinated solvents such as dichloromethane, dichloroethane or mixtures thereof, hydrocarbon solvents such as cyclohexane or heptane. The choice of the solvent is function of the nature of the substrate and/or catalyst and the person skilled in the art is well able to select the solvent most suitable in each case to optimize the reaction.

The elimination step, on the aldehydic substrate, under acidic conditions can be carried out at a temperature in the range comprised between 20° C. and 110° C. Of course, a person skilled in the art is also able to select the preferred temperature according to the melting and boiling point of the starting and final products as well as the desired time of reaction or conversion.

According to a particular embodiment, the elimination, on the aldehydic substrate, may lead to the formation of a mixture comprising different regioisomers which could be isomerised into the desired regioisomer; i.e. compound of formula (I), by protection of the aldehyde functional group in a form of a acetal followed by the isomerisation and deprotection. The protection, isomerisation and deprotection may be performed as reported below.

According to any embodiment of the invention, the process comprises the step of
a) hydroformylation of compound of formula (II) to obtain compound of formula

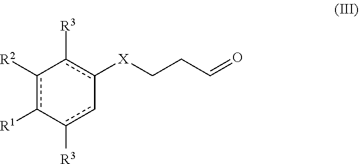

in the form of any one of its stereoisomers or a mixture thereof, and wherein the dotted lines, X, R¹, R² and, R³ have the same meaning as defined above;
b) protection of the aldehyde group of compound formula (III) obtained in step a) in the form of an acetal of formula

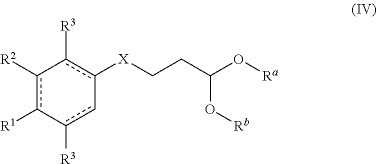

in the form of any one of its stereoisomers or a mixture thereof, and wherein the dotted lines, X, R¹, R², and R³ have the same meaning as defined above and R^{a} and R^{b}, independently from each other, represent a C₁₋₄ alkyl group or R^{a} and R^{b} are taken together and represent a C₂₋₆ alkanediyl group, preferably R^{a} and R^{b} are taken together and represent a (CH₂) n group wherein n is 2 or 3;
c) elimination of the OC(═O) R⁶ group of the compound of formula (IV) followed, optionally by an isomerisation to form a compound of formula

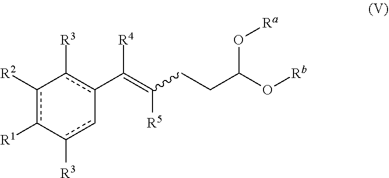

in the form of any one of its stereoisomers or a mixture thereof, and wherein the dotted lines, R¹, R², R³, R⁴ and R⁵ have the same meaning as defined above and R^{a} and R^{b} has the same meaning as defined above; and
d) deprotection of the acetal group to obtain compound of formula (I).

According to any embodiment of the invention, R^{a} and R^{b} may be taken together and represent a C₂₋₆ alkanediyl group. Particularly, R^{a} and R^{b} may be taken together and represent a C₂₋₄ alkanediyl group. Even more particularly, R^{a} and R^{b} are taken together and represent a (CH₂) n group wherein n may be 2 or 3; preferably n may be 2.

According to any embodiment of the invention, the protection of the aldehyde group of compound formula (III) obtained in step a) in the form of an acetal of formula (IV) may be carried out under normal condition known by the person skilled in the art, i.e. with an C₁₋₄ trialkyl orthoformate, C₁₋₄ alcohol and C₂₋₆ diol and in the presence of an acid. Specific and non-limiting examples of acid may be selected from the group consisting of H₂SO₄, KHSO₄, NaHSO₄, H₃PO₄, NaHSO₄, Amberlyst 15, pTsOH, MsOH, TfOH, CSA, oxalic acid, formic acid, TFA, BF₃ Et₂O, BF₃ AcOH, HBF₄, wayphos, SiO₂, Pyridinium p-toluenesulfonate, Zeolite and Al₂(SO₄)₃, F24 X (Clay), boric acid and a mixture thereof.

Specific and non-limiting examples of C₁₋₄ trialkyl orthoformate, C₁₋₄ alcohol and C₂₋₆ diol may be selected from the group consisting of trimethyl orthoformate, triethyl orthoformate, methanol, ethanol, ethylene glycol, 1,2-butanediol, 2,3-butanediol, 2,3-dimethyl-3-hydroxy-2-butanol, diglycerol, trans-1,2-cyclohexandiol, neopentylglycol, 1,3-propanediol, 2-methyl-2-propyl-1,3-propanediol, 1,2-propanediol, 2-methyl-1,2-propanediol, 2,2-dimethyl-1,3-propanediol. Particularly, the acetal formation may be carried out with a C₂₋₆ diol, particularly with ethylene glycol.

The C₁₋₄ trialkyl orthoformate, C₁₋₄ alcohol or C₂₋₆ diol can be added into the reaction medium of the invention's process in a large range of concentrations. As non-limiting examples, one can cite as C₁₋₄ trialkyl orthoformate or C₂₋₅ diol concentration values those ranging from about 1 to about 2 equivalents, relative to the amount of the of substrate. As non-limiting examples, one can cite as C₁₋₄ alcohol concentration values those ranging from about 2 to about 4 equivalents, relative to the amount of the of substrate. The optimum concentration of the C₁₋₄ trialkyl orthoformate, C₁₋₄ alcohol or C₂-6 diol will depend, as the person skilled in the art knows, on the nature of the latter, on the nature of the substrate, on the reaction temperature as well as on the desired time of reaction.

The acid, used in step for protecting of the aldehyde group of formula (III) in the form of an acetal, can be added into the reaction medium of the invention's process in a large range of concentrations. As non-limiting examples, one can cite as acid concentration values those ranging from about 0.1 to about 5 mol %, relative to the amount of the of substrate. The optimum concentration of said acid will depend, as the person skilled in the art knows, on the nature of the latter, on the nature of the substrate, on the reaction temperature as well as on the desired time of reaction.

According to any one of the invention's embodiments, the invention's process to form compound of formula (IV) is carried out at a temperature comprised between 25° C. and 120° C. In particular, the temperature is in the range between 50° C. and 110° C. Of course, a person skilled in the art is also able to select the preferred temperature as a function of the melting and boiling point of the starting and final products as well as the desired time of reaction or conversion.

The acetal formation can be carried out in the presence or absence of a solvent. When a solvent is required or used for practical reasons, then any solvent current in such reaction type can be used for the purposes of the invention. Non-limiting examples include C₆₋₁₂ aromatic solvents such as xylene, toluene, 1,3-diisopropylbenzene, cumene or pseudocumene, or mixtures thereof, hydrocarbon solvents such as cyclohexane, heptane or mixtures thereof. The choice of the solvent is function of the nature of the substrate and/or catalyst and the person skilled in the art is well able to select the solvent most suitable in each case to optimize the reaction.

According to any embodiment of the invention, the elimination of the OC(═O) R⁶ group of the compound of formula (IV), optionally followed by an isomerisation to from a compound of formula (V) may be carried out under normal conditions known by the person skilled in the art, i.e. such as for example pyrolysis followed by isomerisation under acidic conditions or in presence of metal catalyst in elemental form or supported such as Rhodium, Ruthenium, Iridium, Platinum or Palladium complex. The elimination may form the compound of formula (V) and the isomer of formula (V′)

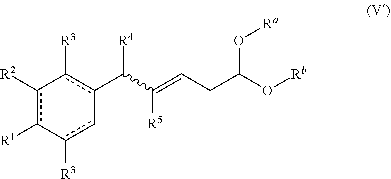

in the form of any one of its stereoisomers or a mixture thereof, and wherein the dotted lines, R¹, R², R³, R⁴, R⁵, R^{a} and R^{b} has the same meaning as defined above
and/or, when R⁵ is not a hydrogen atom, the isomer of formula (V″)

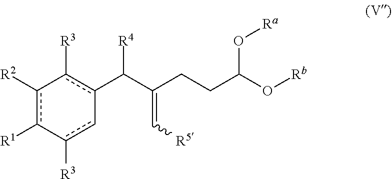

in the form of any one of its stereoisomers or a mixture thereof, and wherein the dotted lines, R¹, R², R³, R⁴, R^{a} and R^{b} has the same meaning as defined above and R^{5′} may be a hydrogen atom or a methyl group.

The isomerisation allows converting the isomer of formula (V′) and the isomer of formula (V″) into the compound of formula (V). Particularly, the elimination and isomerisation may be a one pot process performed in the presence of an acid. The acid may be a Lewis acid or a Bronsted acid. Specific and non-limiting examples of acid may be selected from the group consisting of p-TsOH, MsOH, TfOH, H₂SO₄, H₃PO₄, KHSO₄, NaHSO₄, oxalic acid, formic acid, BF₃ Et₂₀, BF₃ AcOH, Alox acidic (Axsorb A2-5, Al₂O₃ 504C), Amberlyst 15, SiO₂, TFA, Wayphos, polyphosphoric acid, Zeolite (CBV 21A sold by Zeolist, CBV 780 sold by Zeolist, CP814E sold by Zeolist), boric acid, Al₂(SO₄)₃, CSA, Pyridinium p-toluenesulfonate, ZnBr₂, K10-S300 (Bentonite) sold by Clariant, F24 X (Bentonite), Siral® 40 HPV sold by Sasol, HCl, HBr, Zn(SO₄)₂, ZnCl₂, MgI₂ and a mixture thereof.

The acid, used in the one pot elimination/isomerisation reaction, can be added into the reaction medium of the invention's process in a large range of concentrations. As non-limiting examples, one can cite as acid concentration values those ranging from about 1 mol % to about 20 mol %, relative to the amount of the of substrate, preferably from 2 mol % to about 10 mol %, relative to the amount of the of substrate, preferably from about 3 mol % to about 6 mol %, relative to the amount of the of substrate. The optimum concentration of the acid will depend, as the person skilled in the art knows, on the nature of the latter, on the nature of the substrate, on the reaction temperature as well as on the desired time of reaction.

According to any one of the invention's embodiments, the invention's process to form compound of formula (V) is carried out at a temperature comprised between RT and 160° C. In particular, the temperature is in the range between 90° C. and 140° C. Of course, a person skilled in the art is also able to select the preferred temperature as a function of the melting and boiling point of the starting and final products as well as the desired time of reaction or conversion.

The one pot elimination/isomerisation reaction can be carried out in the presence or absence of a solvent. When a solvent is required or used for practical reasons, then any solvent current in such reaction type can be used for the purposes of the invention. Non-limiting examples include C₆₋₁₂ aromatic solvents such as xylene, toluene, 1,3-diisopropylbenzene, cumene or pseudocumene, or mixtures thereof, hydrocarbon solvents such as cyclohexane, heptane or mixtures thereof, esteral or ethereal solvents such as butyl acetate, diisopropyl ether, dioxane, dimethoxyethane or a mixture thereof. The choice of the solvent is function of the nature of the substrate and/or catalyst and the person skilled in the art is well able to select the solvent most suitable in each case to optimize the reaction.

According to a particular embodiment, the protection, elimination and isomerization reactions may be carried out in one pot.

According to any embodiments of the invention, the deprotection of the acetal group to obtain compound of formula (I) may be carried out under normal condition known by the person skilled in the art, i.e. with a large molar excess of carboxylic acid in water. Specific and non-limiting examples of carboxylic acids may be selected from the group consisting of acetic acid, propionic acid, citric acid, formic acid, TFA, oxalic acid or a mixture thereof.

The carboxylic acid, used in the deprotection, can be added into the reaction medium of the invention's process in a large range of concentrations. As non-limiting examples, one can cite as acid concentration values those ranging from about 5 to about 20 equivalents, relative to the amount of the of substrate, preferably from 5 to about 10 equivalents, relative to the amount of the of substrate The optimum concentration of the acid will depend, as the person skilled in the art knows, on the nature of the latter, on the nature of the substrate, on the reaction temperature as well as on the desired time of reaction.

According to any one of the invention's embodiments, the deprotection to form compound of formula (I) may be carried out at a temperature comprised between 40° C. and 120° C. In particular, the temperature is in the range between 70° C. and 90° C. Of course, a person skilled in the art is also able to select the preferred temperature as a function of the melting and boiling point of the starting and final products as well as the desired time of reaction or conversion.

According to any one of the invention's embodiments, the deprotection to form compound of formula (I) can be carried out in the presence or absence of a solvent. When a solvent is required or used for practical reasons, then any solvent current in such reaction type can be used for the purposes of the invention. Non-limiting examples include C₆₋₁₂ aromatic solvents such as toluene, xylene, 1,3-diisopropylbenzene, cumene or pseudocumene, or mixtures thereof, alcoholic solvents such as methanol, ethanol, 2-methylbutan-2-ol or mixtures thereof, hydrocarbon solvents such as cyclohexane, heptane or mixtures thereof, esteric solvents such as n-butyl acetate, iso-propyl acetate, ethyl acetate or ethereal solvents such as methyl tetrahydrofuran, tetrahydrofuran or mixtures thereof. The choice of the solvent is a function of the nature of the substrate and of the carboxylic derivative and the person skilled in the art is well able to select the solvent most convenient in each case to optimize the reaction.

According to a particular embodiment, the protection, elimination, isomerization and deprotection reactions may be carried out in one pot.

The invention's process for the preparation of a compound of formula (I) may be carried out under batch and/or continuous conditions. Particularly, the elimination step may be carried out under continuous conditions.

The compound of formula (III), (IV) and (V) are, generally, novel compounds and present a number of advantages as explained above and shown in the Examples.

Therefore, another object of the present invention is a compound of formula

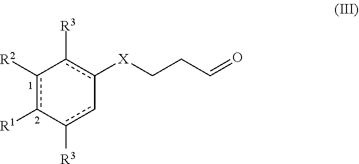

in the form of any one of its stereoisomers or a mixture thereof, and wherein all the dotted lines represent double bonds or the dotted line between carbon 1 and carbon 2 is a carbon-carbon single bond or a carbon-carbon double bond and the other dotted lines are carbon-carbon single bonds; each R¹, R² and R³, independently from each other, represent a hydrogen atom, a C₁₋₃ alkoxy group, a C₁₋₆ alkyl group or a C₂₋₆ alkenyl group, each optionally substituted by a hydroxy or C₁₋₃ alkoxy group; or R¹ and R², are taken together and form a C₃₋₈ cycloalkyl or C₅₋₈ cycloalkenyl group; or both R³, taken together, are a C₁₋₃ alkanediyl group; X represents a CR⁴ (OC(═O) R⁶) CHR⁵ or CHR⁴C(OC(═O) R⁶) (R⁵) wherein R⁴ and R⁵, independently from each other, are a hydrogen atom, a methyl or an ethyl group and R⁶ is a C₁₋₃ alkoxy group, a phenyl group or C₁₋₃ alkyl group optionally substituted by one to three halogen atoms; provided that 5-oxo-1-phenylpentyl acetate is excluded.

Another object of the present invention is compound of formula

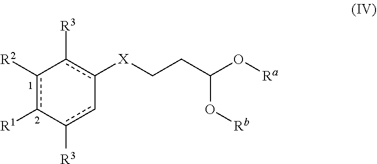

in the form of any one of its stereoisomers or a mixture thereof, and wherein all the dotted lines represent double bonds or the dotted line between carbon 1 and carbon 2 is a carbon-carbon single bond or a carbon-carbon double bond and the other dotted lines are carbon-carbon single bonds; each R¹, R² and R³, independently from each other, represent a hydrogen atom, a C₁₋₃ alkoxy group, a C₁₋₆ alkyl group or a C₂₋₆ alkenyl group, each optionally substituted by a hydroxy or C₁₋₃ alkoxy group; or R¹ and R², are taken together and form a C₃₋₈ cycloalkyl or C₅₋₈ cycloalkenyl group; or both R³, taken together, are a C₁₋₃ alkanediyl group; X represents a CR⁴ (OC (-O) R⁶) CHR⁵ or CHR⁴C(OC(═O) R⁶) (R⁵) wherein R⁴ and R⁵, independently from each other, are a hydrogen atom, a methyl or an ethyl group and R⁶ is a C₁₋₃ alkoxy group, a phenyl group or C₁₋₃ alkyl group optionally substituted by one to three halogen atoms; R^{a} and R^{b}, independently from each other, represent a C₁₋₄ alkyl group or R^{a} and R^{b} are taken together and represent a C₂₋₆ alkanediyl group, preferably R^{a} and R^{b} are taken together and represent a (CH₂) n group wherein n is 2 or 3.

Another object of the present invention is a compound of formula

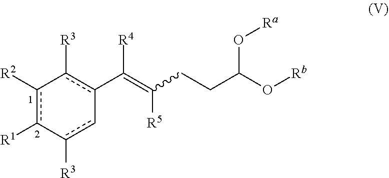

in the form of any one of its stereoisomers or a mixture thereof, and wherein all the dotted lines represent double bonds or the dotted line between carbon 1 and carbon 2 is a carbon-carbon single bond or a carbon-carbon double bond and the other dotted lines are carbon-carbon single bonds; each R¹, R² and R³, independently from each other, represent a hydrogen atom, a C₁₋₃ alkoxy group, a C₁₋₆ alkyl group or a C₂₋₆ alkenyl group, each optionally substituted by a hydroxy or C₁₋₃ alkoxy group; or R¹ and R², are taken together and form a C₃₋₈ cycloalkyl or C₅₋₈ cycloalkenyl group; or both R³, taken together, are a C₁₋₃ alkanediyl group; R⁴ and R⁵, independently from each other, are a hydrogen atom, a methyl or an ethyl group; R^{a} and R^{b}, independently from each other, represent a C₁₋₄ alkyl group or R^{a} and R^{b} are taken together and represent a C₂₋₆ alkanediyl group, preferably R^{a} and R^{b} are taken together and represent a (CH₂) n group wherein n is 2 or 3; provided that 2-(4-cyclohexylbut-3-en-1-yl)-1,3-dioxolane, 2-(4-phenylbut-3-en-1-yl)-1,3-dioxolane and 2-(4-phenylpent-3-en-1-yl)-1,3-dioxolane are excluded.

Another object of the present invention is the use of a compound of formula

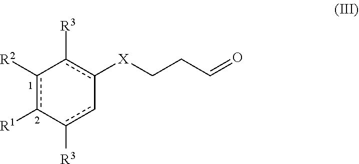

in the form of any one of its stereoisomers or a mixture thereof, and wherein all the dotted lines represent double bonds or the dotted line between carbon 1 and carbon 2 is a carbon-carbon single bond or a carbon-carbon double bond and the other dotted lines are carbon-carbon single bonds; each R¹, R² and R³, independently from each other, represent a hydrogen atom, a C₁₋₃ alkoxy group, a C₁₋₆ alkyl group or a C₂₋₆ alkenyl group, each optionally substituted by a hydroxy or C₁₋₃ alkoxy group; or R¹ and R², are taken together and form a C₃₋₈ cycloalkyl or C₅₋₈ cycloalkenyl group; or both R³, taken together, are a C₁₋₃ alkanediyl group; X represents a CR⁴(OC(═O)R⁶)CHR⁵ or CHR⁴C(OC(═O)R⁶)(R⁵) wherein R⁴ and R⁵, independently from each other, are a hydrogen atom, a methyl or an ethyl group and R⁶ is a C₁₋₃ alkoxy group, a phenyl group or C₁₋₃ alkyl group optionally substituted by one to three halogen atoms;
in the preparation of compound of formula (I).

Another object of the present invention is the use of a compound of formula

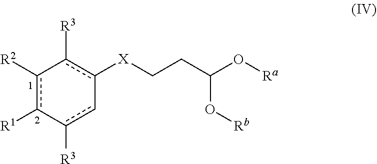

in the form of any one of its stereoisomers or a mixture thereof, and wherein all the dotted lines represent double bonds or the dotted line between carbon 1 and carbon 2 is a carbon-carbon single bond or a carbon-carbon double bond and the other dotted lines are carbon-carbon single bonds; each R¹, R² and R³, independently from each other, represent a hydrogen atom, a C₁₋₃ alkoxy group, a C₁₋₆ alkyl group or a C₂₋₆ alkenyl group, each optionally substituted by a hydroxy or C₁₋₃ alkoxy group; or R¹ and R², are taken together and form a C₃₋₈ cycloalkyl or C₅₋₈ cycloalkenyl group; or both R³, taken together, are a C₁₋₃ alkanediyl group; X represents a CR⁴(OC(═O)R⁶) CHR⁵ or CHR⁴C(OC(═O)R⁶)(R⁵) wherein R⁴ and R⁵, independently from each other, are a hydrogen atom, a methyl or an ethyl group and R⁶ is a C₁₋₃ alkoxy group, a phenyl group or C₁₋₃ alkyl group optionally substituted by one to three halogen atoms; R^{a} and R^{b}, independently from each other, represent a C₁₋₄ alkyl group or R^{a} and R^{b} are taken together and represent a C₂₋₆ alkanediyl group, preferably R^{a} and R^{b} are taken together and represent a (CH₂) n group wherein n is 2 or 3;
in the preparation of compound of formula (I).

Another object of the present invention is the use of a compound of formula

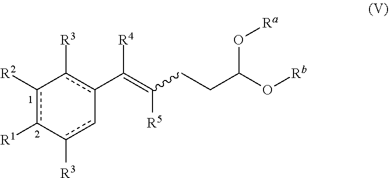

in the form of any one of its stereoisomers or a mixture thereof, and wherein all the dotted lines represent double bonds or the dotted line between carbon 1 and carbon 2 is a carbon-carbon single bond or a carbon-carbon double bond and the other dotted lines are carbon-carbon single bonds; each R¹, R² and R³, independently from each other, represent a hydrogen atom, a C₁₋₃ alkoxy group, a C₁₋₆ alkyl group or a C₂₋₆ alkenyl group, each optionally substituted by a hydroxy or C₁₋₃ alkoxy group; or R¹ and R², are taken together and form a C₃₋₈ cycloalkyl or C₅₋₈ cycloalkenyl group; or both R³, taken together, are a C₁₋₃ alkanediyl group; R⁴ and R⁵, independently from each other, are a hydrogen atom, a methyl or an ethyl group; R^{a} and R^{b}, independently from each other, represent a C₁₋₄ alkyl group or R^{a} and R^{b} are taken together and represent a C₂₋₆ alkanediyl group, preferably R^{a} and R^{b} are taken together and represent a (CH₂) n group wherein n is 2 or 3;
in the preparation of compound of formula (I).

Typical manners to execute the invention's process are reported herein below in the examples.

### EXAMPLES

The invention will now be described in further detail by way of the following examples, wherein the abbreviations have the usual meaning in the art, the temperatures are indicated in degrees centigrade (° C.). The preparation of precatalysts and ligands solutions were carried out under an inert atmosphere (Argon) using standard Schlenk techniques. The solvents were dried by conventional procedures and distilled under an argon atmosphere. NMR spectra were recorded at 20° C. on Bruker AV 300, AV 400, or AV 500 MHz spectrometers. Chemical shifts are reported in ppm relative to solvent signals (chloroform, 8H=7.26 ppm, 8c=77.0 ppm). The signal assignment was ensured by recording 1H, 1H-COSY,-NOESY, 13C,1H-HSQC and -HMBC experiments. Gas chromatography was performed on an Agilent 7890 A Series equipped with a HP5 column (30 m×0.25 mm ID, 0.25 μm film) and tetradecane was used as internal standard.

### Example 1

Preparation of 2-methyl-1-(p-tolyl) but-3-en-2-ol from 4-methylphenylacetone

To a cooled solution (0° C.) of 139.9 mL vinylmagnesium chloride (1.6 M in THF, 254.4 mmol, 1.1 eq) was added slowly a solution of 1-(p-tolyl) propan-2-one (37.7 g, 254.4 mmol) in 153 mL THF. The internal temperature did not exceed 5° C. during the addition of the ketone. The mixture was further stirred at 0° C. over night (16 hours). The reaction mixture was added slowly to a cooled solution of 18.3 g AcOH (305.2 mmol) in 200 ml water. The phases were separated and the aqueous phase was extracted with 150 mL TBME. The combined organic phase were washed with a saturated aqueous NaHCO₃ solution and a saturated aqueous NaCl solution. After drying over Na₂SO₄ the solvent was evaporated under reduced pressure (500-20 mbar, 50° C.). The crude (48.2 g) was purified by a distillation through a Vigreux column under reduced pressure (oilbath 40° C.-125° C., 50-4 mbar, bp 97° C./4 mbar). 35.5 g (201.4 mmol, 79% yield) 2-methyl-1-(p-tolyl) but-3-en-2-ol of a colourless liquid were obtained.

2-methyl-1-(p-tolyl) but-3-en-2-ol:

¹H-NMR analysis results in CDCl₃ were in accordance with data from literature: Araki, S.; Ohmura, M.; Butsugan, Y. Bulletin of the Chemical Society of Japan (1986), 59 (6), 2019-20.

¹³C NMR (100 MHz, CDCl₃): δ21.0, 27.4, 48.3, 73.0, 111.9, 128.8, 130.5, 133.7, 136.1, 144.8.

### Example 2

Preparation of 2-methyl-1-(p-tolyl) but-3-en-2-yl acetate from 2-methyl-1-(p-tolyl) but-3-en-2-ol

To a stirred solution of 2-methyl-1-(p-tolyl) but-3-en-2-ol (5 g, 28.37 mmol) and Acetic anhydride (5.8 g, 56.7 mmol) in Toluene (5 mL) was added DMAP (173 mg, 1.42 mmol, 5 mol %) and triethylamine (2.87 g, 28.37 mmol) under N₂. The mixture was heated at 80° C. for 5 hours. The mixture was cooled with a cold-water bath (10° C.) and 5 mL of water were added slowly (hydrolysis of residual Ac₂O). 6.97 g of an aqueous 25% NaOH solution (1.5 eq) were added slowly. After stirring for 30 min, 20 mL of MTBE were added. The phases were separated, and the organic phase was washed twice with water (10 mL), and then once with a saturated aqueous NaHCO₃ solution (15 mL). After a final wash with brine (10 mL) the organic phase was dried over sodium sulfate, filtered and evaporated under reduced pressure (45° C., 30 mbar). The crude (6.1 g) was purified by flash chromatography (120 g cartridge, eluent from pentane 100% to pentane 95/MTBE 5). 2-methyl-1-(p-tolyl) but-3-en-2-yl acetate was isolated (5.5 g, 25.196 mmol, 88.8 yield) as a colourless liquid.

2-methyl-1-(p-tolyl) but-3-en-2-yl acetate

¹H-NMR (500.15 MHz): δ1.51 (s, 3H), 1.99 (s, 3H), 2.32 (s, 3H), 2.32 (s, 3H), 2.46 (m, 2H), 2.99 (d, 1H, J=13.6 Hz, 3.11 (d, 1H, 13.6 Hz), 5.11 (d, 1H, J=10.8 Hz), 5.13 (d, 1H, J=3.9 Hz), 6.02 (dd, 1H, J=17.5 Hz, J=11.1 Hz) 7.05-7.11 (m, 4H).

¹³C NMR (125 MHz, CDCl₃): δ 21.1, 22.3, 23.3, 45.5, 82.6, 113.5, 128.6, 130.7, 133.3, 136.0, 141.7, 170.1.

### Example 3

Hydroformylation of 2-methyl-1-(p-tolyl) but-3-en-2-yl acetate.

The autoclave was charged with 2-methyl-1-(p-tolyl) but-3-en-2-yl acetate (5.01 g, 22.951 mmol), Rh(CO) zacac (3.0 mg, 0.0116 mmol) and BiPhePhos (27.3 mg, 0.0347 mmol). The vessel was purged with H₂/CO (1:1, 4×5 bar) and heated under vigorous stirring at 90° C. and 10 bar syngas pressure for 24 h. After cooling and depressurization, GLC analysis of the crude yellow oil revealed total conversion and the presence of the linear 2-methyl-5-oxo-1-(p-tolyl) pentan-2-yl acetate (89%), hydrogenated starting material 2-methyl-1-(p-tolyl) butan-2-yl acetate (8%) and some minor unidentified side-products. This crude (5 g) reaction mixture was further processed in the next stage.

2-methyl-5-oxo-1-(p-tolyl) pentan-2-yl acetate:

¹H-NMR (500.15 MHz): δ 1.41 (s, 3H), 1.93-2.0 (m, 1H), 1.98 (s, 3H), 2.22-2.29 (m, 1H), 2.32 (s, 3H), 2.46 (m, 2H), 3.1 (q, 2H), 7.01-7.1 (m, 4H), 9.73 (t, 1H).

¹³C NMR (125 MHZ, CDCl₃): δ21.0 (q), 22.4 (q), 23.5 (q), 30.8 (t), 38.7 (t), 43.8 (t), 83.3 (s), 128.9 (d), 130.3 (d), 133.4 (s), 136.2 (s), 170.6 (s), 201.7 (d).

### Example 4

Preparation of 4-(1,3-dioxolan-2-yl)-2-methyl-1-(p-tolyl) butan-2-yl acetate from 2-methyl-5-oxo-1-(p-tolyl) pentan-2-yl acetate

The crude (GC 89%, 5 g) of the previous experiment (contains 19.5 mmol of 2-methyl-5-oxo-1-(p-tolyl) pentan-2-yl acetate) was stirred in the presence of 1.8 g (29.25 mmol, 1.5 eq)ethylene glycol and 133 mg KHSO₄ (0.975 mmol, 5 mol %) under Dean-Stark conditions in 5 mL toluene at 105-113° C. for 1 h (internal temperature, water was eliminated during 1 hour). The mixture was cooled down to room temperature and 15 mL TBME were added. After washing with 5 mL water, 5 mL of a saturated aqueous NaHCO₃ solution and 5 ml of brine the organic phase was dried over Na₂SO₄ and the solvent was evaporated under reduced pressure. The crude (GC 86%) was purified by column chromatography (120 g cartridge, from cyclohexane/MTBE 97/3 to cyclohexane/MTBE 7/3). 5.13 g (17.55 mmol) 4-(1,3-dioxolan-2-yl)-2-methyl-1-(p-tolyl) butan-2-yl acetate were isolated as a colourless liquid (76% yield over 2 steps).

¹H-NMR (500.15 MHZ):): δ 1.38 (s, 3H), 1.68-1.85 (m, 3H), 1.97 (s, 3H), 2.00-2.07 (m, 1H), 2.32 (s, 3H), 2.99 (d, 1H, J=13.7 Hz), 3.17 (d, 1H, J=13.7 Hz), 3.81-3.88 (m, 2H). 3.92-4.00 (m, 2H), 4.84 (t, 1H, J=4.5 Hz), 7.04-7.11 (m, 4H).

¹³C NMR (125 MHz, CDCl₃): δ21.0, 22.4, 23.6, 28.4, 32.5, 43.7, 64.9, 83.8, 104.5, 128.7, 130.4, 133.8, 136.0, 170.6.

### Example 5

Preparation of (E)-2-(3-methyl-4-(p-tolyl) but-3-en-1-yl)-1,3-dioxolane from 4-(1,3-dioxolan-2-yl)-2-methyl-1-(p-tolyl) butan-2-yl acetate

### a) Under Termal Pyrolysis

2 g (6.84 mmol) 4-(1,3-dioxolan-2-yl)-2-methyl-1-(p-tolyl) butan-2-yl acetate in 20 mL heptane were added slowly (12.5 mL/h) from the top and under a N₂ flow to a heated pyrolysis column (pyrolysis oven at 500° C.), which was filled with 20 g quartz cylinder (Raschig 4×3 mm). When the addition was finished, the oven was cooled down and 5 ml heptane were added to wash the quartz cylinder. After the addition of further 5 mL of heptane the mixture was washed with 5 mL water, with 5 mL of a saturated aqueous NaHCO₃ solution and with 5 mL water. The aqueous phases were combined and extracted once with 10 mL of cyclohexane. The combined organic phase was washed with brine and was dried over sodium sulfate. The solvent was evaporated under reduced pressure (Rotavap 10 mbar, 50° C.). 1.40 g (6.03 mmol, 88% yield) of a mixture of unsaturated dioxolanes comprising (E)-2-(3-methyl-4-(p-tolyl) but-3-en-1-yl)-1,3-dioxolane, (Z)-2-(3-methyl-4-(p-tolyl) but-3-en-1-yl)-1,3-dioxolane, (E/Z)-2-(3-methyl-4-(p-tolyl) but-2-en-1-yl)-1,3-dioxolane, 2-(3-(4-methylbenzyl) but-3-en-1-yl)-1,3-dioxolane (ratio 29.4/24.1/17.7/28.8) were obtained after a column filtration.

The quantity of (E)-2-(3-methyl-4-(p-tolyl) but-3-en-1-yl)-1,3-dioxolane and (Z)-2-(3-methyl-4-(p-tolyl) but-3-en-1-yl)-1,3-dioxolane can be enriched by heating the mixture of isomers (E)-2-(3-methyl-4-(p-tolyl) but-3-en-1-yl)-1,3-dioxolane/(Z)-2-(3-methyl-4-(p-tolyl) but-3-en-1-yl)-1,3-dioxolane/(E/Z)-2-(3-methyl-4-(p-tolyl) but-2-en-1-yl)-1,3-dioxolane/2-(3-(4-methylbenzyl) but-3-en-1-yl)-1,3-dioxolane (6.03 mmol, ratio 29.4/24.1/17.7/28.8) in the presence of 6 mol % pTsOH and toluene (110° C.). After heating 4 hours at 110° C. a ratio 45/32/20/3 (E)-2-(3-methyl-4-(p-tolyl) but-3-en-1-yl)-1,3-dioxolane/(Z)-2-(3-methyl-4-(p-tolyl) but-3-en-1-yl)-1,3-dioxolane/(E/Z)-2-(3-methyl-4-(p-tolyl) but-2-en-1-yl)-1,3-dioxolane/2-(3-(4-methylbenzyl) but-3-en-1-yl)-1,3-dioxolane was observed. The quantity of (E)-2-(3-methyl-4-(p-tolyl) but-3-en-1-yl)-1,3-dioxolane was increased from 1.77 mmol to 2.71 mmol in 4 hours (40% yield over 2 steps). The quantity of (Z)-2-(3-methyl-4-(p-tolyl) but-3-en-1-yl)-1,3-dioxolane was increased from 1.45 mmol to 1.93 mmol in 4 hours (40+28% yield over 2 steps for both isomers)

(E)-2-(3-methyl-4-(p-tolyl) but-3-en-1-yl)-1,3-dioxolane:

¹H-NMR (500.15 MHz): 1.86 (s, 3H), 1.87-1.90 (m, 2H), 2.26-2.30 (m, 2H), 2.34 (s, 3H), 3.83-3.90 (m, 2H), 3.96-4.01 (m, 2H), 4.92 (t, 1H, J=4.7 Hz), 6.27 (s, 1H), 7.08-7.15 (m, 4H).

¹³C NMR (125 MHz, CDCl₃): δ 17.8, 21.1, 32.4, 34.9, 64.9, 104.2, 124.9, 128.7, 128.7, 135.5, 135.5, 137.3.

GC 4.49 min (column DB-1 10 m, 80° C.-320° C. (30° C./min)

(Z)-2-(3-methyl-4-(p-tolyl) but-3-en-1-yl)-1,3-dioxolane (signals from the mixture):

¹H-NMR (500.15 MHZ): 1.88 (s, 3H), 1.84-1.86 (m, 2H), 2.32 (s, 3H), 2.34-2.38 (m, 2H), 3.80-3.86 (m, 2H), 3.92-3.98 (m, 2H), 4.87 (t, 1H, J=4.6 Hz), 6.26 (s, 1H), 7.11-7.15 (m, 4H).

¹³C NMR (125 MHZ, CDCl₃): δ21.1, 24.1, 26.9, 32.3, 64.9, 104.3, 125.9, 128.4, 128.8,135.3, 135.5, 137.7.

GC 4.25 min (column DB-1 10 m, 80° C.-320° C. (30° C./min)

(E)-2-(3-methyl-4-(p-tolyl) but-2-en-1-yl)-1,3-dioxolane (characteristic signals):

¹H-NMR (500.15 MHZ): 2.31 (s, 3H), 2.41-2.45 (m, 2H), 3.28 (s, 2H).

¹³C NMR (125 MHz, CDCl₃): δ 104.3, 119.28.

GC 4.23 min (column DB-1 10 m, 80° C.-320° C. (30° C./min)

(Z)-2-(3-methyl-4-(p-tolyl) but-2-en-1-yl)-1,3-dioxolane (characteristic signals):

¹H-NMR (500.15 MHZ): 3.35 (s, 2H).

¹³C NMR (125 MHz, CDCl₃): δ 104.3, 119.25.

GC 4.23 min (column DB-1 10 m, 80° C.-320° C. (30° C./min)

2-(3-(4-methylbenzyl) but-3-en-1-yl)-1,3-dioxolane (characteristic signals):

¹H-NMR (500.15 MHZ): 3.30 (s, 2H), 4.75 (s, 1H), 4,83 (s, 1H).

¹³C NMR (125 MHz, CDCl₃): δ 104.2, 111.0, 148.3.

GC 4.18 min (column DB-1 10 m, 80° C.-320° C. (30° C./min)

### b) Under Acidic Conditions

123 mg (0.855 mmol, 5 mol %) pTsOH H₂O were heated under stirring and Dean-Stark conditions (reflux) at 110° C. for 30 min in 15 mL toluene. 5.0 g (17.1 mmol) 4-(1,3-dioxolan-2-yl)-2-methyl-1-(p-tolyl) butan-2-yl acetate mL toluene, were added slowly (15 min). The mixture was further stirred for 2.5 hours at 110° C. After cooling down to room temperature 25 ml of cyclohexane were added. After washing twice with 25 mL of a saturated aqueous NaHCO₃ solution, with 25 ml of water and with 25 ml of brine the organic phase was dried over Na₂SO₄ and the solvent was evaporated under reduced pressure (50° C., crude 4.03 g, GC ratio 40/27/27/6 (E)-2-(3-methyl-4-(p-tolyl) but-3-en-1-yl)-1,3-dioxolane/(Z)-2-(3-methyl-4-(p-tolyl) but-3-en-1-yl)-1,3-dioxolane/(E/Z)-2-(3-methyl-4-(p-tolyl) but-2-en-1-yl)-1,3-dioxolane/2-(3-(4-methylbenzyl) but-3-en-1-yl)-1,3-dioxolane, 94% yield (16.0 mmol) of all isomers of unsaturated dioxolanes, 6.4 mmol (E)-2-(3-methyl-4-(p-tolyl) but-3-en-1-yl)-1,3-dioxolane, 4.32 mmol (Z)-2-(3-methyl-4-(p-tolyl) but-3-en-1-yl)-1,3-dioxolane, 37+25% yield).

The quantity of (E)-2-(3-methyl-4-(p-tolyl) but-3-en-1-yl)-1,3-dioxolane and (Z)-2-(3-methyl-4-(p-tolyl) but-3-en-1-yl)-1,3-dioxolane can be further enriched by heating the mixture of isomers (E)-2-(3-methyl-4-(p-tolyl) but-3-en-1-yl)-1,3-dioxolane/(Z)-2-(3-methyl-4-(p-tolyl) but-3-en-1-yl)-1,3-dioxolane/(E/Z)-2-(3-methyl-4-(p-tolyl) but-2-en-1-yl)-1,3-dioxolane/2-(3-(4-methylbenzyl) but-3-en-1-yl)-1,3-dioxolane in the presence of 6 mol % pTsOH and toluene (110° C.): 7.2 mmol (E)-2-(3-methyl-4-(p-tolyl) but-3-en-1-yl)-1,3-dioxolane, 5.12 mmol (Z)-2-(3-methyl-4-(p-tolyl) but-3-en-1-yl)-1,3-dioxolane, 42+30% yield.

### Example 6

Preparation of 2-methyl-1-(p-tolyl) but-3-en-1-ol from 4-methylbenzaldehyde

To a cooled solution (0° C.) of 494.4 mL 1-methyl-2-propenylmagnesium chloride (0.5 M in THF, 247.2 mmol, 1.1 eq) was added slowly a solution of 4-methylbenzaldehyde (27.0 g, 224.7 mmol) in 135 mL THF. The internal temperature did not exceed 5° C. during the addition of the aldehyde. The mixture was further stirred at 0° C. overnight (16 hours) and analysed by GC. The reaction mixture was added slowly to a cooled solution of 16.2 g AcOH (269.7 mmol) in 200 ml water. The phases were separated and the aqueous phase was extracted twice with 150 mL TBME. The combined organic phase were washed with a saturated aqueous NaHCO₃ solution and a saturated aqueous NaCl solution. After drying over Na₂SO₄ the solvent was evaporated under reduced pressure (500-4 mbar, 50° C.). The crude (44.3 g) was purified by a distillation through a Vigreux column under reduced pressure (oil bath 120° C., 900-3 mbar, bp 90° C./3 mbar). 39.0 g (221.3 mmol, 98.4% yield) 2-methyl-1-(p-tolyl) but-3-en-1-ol (syn/anti mixture) of a colourless liquid were obtained.

NMR analysis results in CDCl₃ were in accordance with data from literature for the syn isomer:

Shibata, I.; Yoshimura, N.; Yabu, M. Baba, A. Eur. J. Org. Chem. 2001, 3207-3211.

S. Hayashi, K. Hirano, H. Yorimitsu, K. Oshima Org. Lett. 2005, 7, 16, 3577-3579.

2-methyl-1-(p-tolyl) but-3-en-1-ol (syn isomer):

¹³C NMR (125 MHz, CDCl₃): δ14.2, 21.1, 44.6, 77.2, 115.4, 126.5, 128.8, 137.0, 139.6, 140.4.

2-methyl-1-(p-tolyl) but-3-en-1-ol (anti isomer):

¹³C NMR (125 MHz, CDCl₃): δ16.6, 21.1, 46.2, 77.7, 116.7, 126.8, 128.9, 137.3, 139.5, 140.8.

### Example 7

Preparation of 2-methyl-1-(p-tolyl) but-3-en-1-yl acetate from 2-methyl-1-(p-tolyl) but-3-en-1-ol

To 9.94 g (56.397 mmol) 2-methyl-1-(p-tolyl) but-3-en-1-ol was added DMAP (172 mg, 1.41 mmol, 2.5 mol %) and triethylamine (5.71 g, 56.4 mmol, 1 eq) under stirring and N₂ atmosphere. Then Acetic anhydride (11.515 g, 112.79 mmol, 2 eq) was added slowly (exothermic). The mixture was stirred 1.5 hours at room temperature (complete conversion). The mixture was cooled with an ice bath (0° C.) and 5 mL of water were added slowly (hydrolysis of residual Ac₂O). 13.8 g of an aqueous 25% NaOH solution (1.5 eq) were added slowly. After stirring for 30 min, 25 mL of MTBE were added. The phases were separated. The aqueous phase was extracted once with 25 mL MTBE. The combined organic phases were washed twice with water (15 mL), and then once with a saturated aqueous NaHCO₃ solution (15 mL) and once with water (15 mL). After a final wash with brine (10 mL) the organic phase was dried over sodium sulfate, filtered and evaporated under reduced pressure (50° C., 50 mbar). The crude (12.3 g) was purified by flash chromatography (220 g cartridge, eluent from pentane 100% to pentane 95/MTBE 5). 2-methyl-1-(p-tolyl) but-3-en-2-yl acetate (1/1 mixture of diastereomers) was isolated (11.93 g, 54.64 mmol, 97 yield) as a colourless liquid.

2-methyl-1-(p-tolyl) but-3-en-2-yl acetate (1/1 mixture of diastereomers)

¹H-NMR (500.15 MHz): δ 0.87 (d, 1.5H, J=6.9 Hz), 1.05 (d, 1.5H, 6.9 Hz) 2.02 (s, 1.5H), 2.07 (s, 1.5H), 2.32 (s, 1.5H), 2.33 (s, 1.5H), 2.61-2.70 (m, 1H), 4.92-4.95 (m,

0.5H), 4.96-5.05 (m, 1H), 5.01-5.08 (m, 0.5H), 5.57 (dd, ¹H, J=18.3 Hz, J=8.1 Hz), 5.61-5.68 (m, 0.5H), 5.71-5.80 (m, 0.5H), 7.10-7.22 (m, 4H).

¹³C NMR (125 MHz, CDCl₃): δ 16.4, 21.2, 21.2, 43.5, 78.9, 115.5, 127.2, 128.9, 136.1, 137.6, 140.0, 170.2.

¹³C NMR (125 MHz, CDCl₃): δ 15.4, 21.1, 21.1, 42.7, 78.9, 115.5, 127.1, 128.8, 136.0, 137.4, 139.2, 170.2.

### Example 8

Hydroformylation of 2-methyl-1-(p-tolyl) but-3-enyl acetate, racemic 1:1 diastereomeric mixture.

The autoclave was charged with a racemic diastereomeric mixture of 2-methyl-1-(p-tolyl)-but-3-enyl acetates (5.04 g, 23.0089 mmol), Rh(CO) zacac (3.1 mg, 0.012 mmol) and 6,6′-((3,3′-di-tert-butyl-5,5′-dimethoxy-[1,1′-biphenyl]-2,2′-diyl)bis(oxy)) didibenzo[d,f] [1,3,2] dioxaphosphepine, abbreviated to BiPhePhos (27.9 mg, 0.0355 mmol). The vessel was purged with H₂/CO (1:1, 4×5 bar) and heated under vigorous stirring at 90° C. and 10 bar syngas pressure for 24 h. After cooling and depressurization, GLC analysis of the crude colorless oil revealed total conversion and the presence of the linear racemic 1:1 diastereomeric 2-methyl-5-oxo-1-(p-tolyl) pentyl acetates, (85%, 82% yield; no base line separation), hydrogenated starting material (7.1%, no base line separation) and two unidentified products (1.4%/5.5%), which are presumably isomerized starting material. This crude reaction mixture (5.5 g) was further processed in the next stage.

2-methyl-5-oxo-1-(p-tolyl) pentyl acetates (˜1:1 diast.mix):

¹H-NMR (500.15 MHz): δ 0.78 (d, 3H), 0.94 (d, 3H), 1.38-1.41 (m, 1H), 1.42-1.51 (m, 1H), 1.62-1.72 (m, 2H), 1.85-2.03 (m, 3H), 2.06 (s, 3H), 2.08 (s, 3H), 2.33 (s, 6H), 2.36-2.56 (m, 3H), 5.51 (d, 1H), 5.59 (d, 1H), 7.1-7.2 (m, 8H), 9.69 (s, 1H), 9.76 (s, 1H).

¹³C NMR (125 MHz, CDCl₃): δ 15.0 (q), 21.1 (q), 21.1 (q), 24.5 (t), 37.9 (d), 41.4 (t), 78.9 (d), 126.8 (d), 129.0 (d), 136.2 (s), 137.6 (s), 170.3 (s), 202.1 (d).

### Example 9

Preparation of 4-(1,3-dioxolan-2-yl)-2-methyl-1-(p-tolyl)butyl acetate from 2-methyl-5-oxo-1-(p-tolyl) pentyl acetates

The 4.5 g of the crude (GC 85%) of the previous experiment (contains 15.4 mmol of 2-methyl-5-oxo-1-(p-tolyl) pentyl acetates, 1/1 mixture of diasteriomers) was stirred in the presence of 1.51 g (23.1 mmol, 1.5 eq) ethylene glycol and 105 mg KHSO₄ (0.770 mmol, 5 mol %) under Dean-Stark conditions in 4.5 mL toluene at 105-113° C. for 1 h (internal temperature, water was eliminated during 1 hour). The mixture was cooled down to room temperature and 15 mL TBME were added. After washing with 5 mL water, 5 mL of a saturated aqueous NaHCO₃ solution and 5 ml of brine the organic phase was dried over Na₂SO₄ and the solvent was evaporated under reduced pressure. The crude (5.7 g, GC 84.4%) was purified by column chromatography (120 g cartridge, from cyclohexane/MTBE 97/3 to cyclohexane/MTBE 7/3). 4.0 g (13.681 mmol) 4-(1,3-dioxolan-2-yl)-2-methyl-1-(p-tolyl) butyl acetate (1/1 mixture of diasteriomers) were isolated as a colourless liquid (89% yield, 73% yield over 2 steps).

4-(1,3-dioxolan-2-yl)-2-methyl-1-(p-tolyl) butyl acetate (1/1 mixture of diasteriomers)

¹H-NMR (500.15 MHZ): δ 0.77 (d, 1.5H, J=6.8 Hz), 0.93 (d, 1.5H, 6.8 Hz) 1.12-1.28 (m, 1H), 1.43-1.50 (m, 0.5H), 1.54-1.80 (m, 1.5H), 1.89-2.03 (1H), 2.05 (s, 1.5H), 2.07 (s, 1.5H), 2.32 (s, 3H), 3.78-3.86 (m, 2H). 3.90-3.97 (m, 2H), 4.76 (t, 1H, J=4.8 Hz), 4.83 (t, 1H, J=4.7 Hz), 5.49 (d, 0.5H, J=7.7 Hz), 5.60 (d, 0.5H, J=6.4 Hz), 7.10-7.20 (m, 4H).

¹³C NMR (125 MHz, CDCl₃): δ 14.9, 21.1, 21.2, 27.0, 31.4, 38.0, 64.8, 64.8, 79.2, 104.7, 126.7, 128.9, 136.6, 137.4, 170.3.

¹³C NMR (125 MHz, CDCl₃): δ 15.4, 21.1, 21.3, 26.6, 31.3, 38.4, 64.8, 64.9, 79.9, 104.6, 127.1, 128.9, 136.4, 137.2, 170.3.

### Example 10

Preparation of (E)-2-(3-methyl-4-(p-tolyl) but-3-en-1-yl)-1,3-dioxolane and (Z)-2-(3-methyl-4-(p-tolyl) but-3-en-1-yl)-1,3-dioxolane from 4-(1,3-dioxolan-2-yl)-2-methyl-1-(p-tolyl) butan-2-yl acetate

A mixture of 570 mg (1.95 mmol) 4-(1,3-dioxolan-2-yl)-2-methyl-1-(p-tolyl) butan-2-yl acetate (1/1 mixture of diasteriomers) and 130 mg heptane were added slowly (12 mL/h) from the top and under a N₂ flow to a heated pyrolysis column (pyrolysis oven at 500° C.), which was filled with 20 g quartz cylinder (Raschig 4*3 mm). When the addition was finished, the oven was cooled down and 5 ml heptane were added to wash the quartz cylinder. After the addition of further 5 mL of heptane the mixture was washed with 5 mL water, with 5 mL of a saturated aqueous NaHCO₃ solution and with 5 mL water. The aqueous phases were combined and extracted once with 10 mL of heptane. The combined organic phase was washed with brine and was dried over sodium sulfate. The solvent was evaporated under reduced pressure (Rotavap 10 mbar, 50° C.).

A product mixture (433 mg) comprising 88 mg (0.300 mmol, 15% yield) 4-(1,3-dioxolan-2-yl)-2-methyl-1-(p-tolyl) butan-2-yl acetate (1/1 mixture of diasteriomers, recycled starting material), 190 mg (0.819 mmol, 42% yield) (E)-2-(3-methyl-4-(p-tolyl) but-3-en-1-yl)-1,3-dioxolane, 155 mg (0.667 mmol, 34% yield) (Z)-2-(3-methyl-4-(p-tolyl) but-3-en-1-yl)-1,3-dioxolane could be isolated.

### Example 11

Preparation of 4-methyl-5-(p-tolyl) pent-4-enal from 2-(3-methyl-4-(p-tolyl) but-3-en-1-yl)-1,3-dioxolane

9.6 g (41.32 mmol) 2-(3-methyl-4-(p-tolyl) but-3-en-1-yl)-1,3-dioxolane 14.5 mL heptane, 15 g water and 15 g AcOH (5 eq) were mixed and heated under stirring at 80° C. for 4 hours (63% conversion). The mixture was cooled down to 0° C.-5° C. and a 26% aqueous NaOH solution (9 g NaOH, 25 g water) was added in 1.5 hours (internal temperature under 10° C.). The aqueous phase was separated (pH 6) and reextracted with 15 mL heptane. The combined organic phases were washed with 10 mL water and 15 mL of a saturated aqueous NaHCO₃ solution. The organic phase was dried over Na₂SO₄ and the solvent was evaporated under reduced pressure. 8.73 g of a mixture of 4-methyl-5-(p-tolyl) pent-4-enal (GC 63%, 29.2 mmol, 71% yield) and 2-(3-methyl-4-(p-tolyl) but-3-en-1-yl)-1,3-dioxolane (GC 32%, 12.0 mmol, 29% yield) were obtained (99% yield on 5 conversion).

4-methyl-5-(p-tolyl) pent-4-enal

The 1H and 13C NMR analysis results in CDCl₃ were in accordance with data from literature (see R. Moretti WO 2010052635 A1).

1. A process for the preparation of preparing a compound of formula 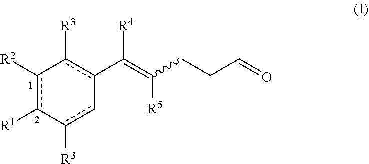
in the form of any one of its stereoisomers or a mixture thereof, wherein all the dotted lines represent double bonds or the dotted line between carbon 1 and carbon 2 is a carbon-carbon single bond or a carbon-carbon double bond and the other dotted lines are carbon-carbon single bonds; each R¹, R² and R³, independently from each other, represent a hydrogen atom, a C₁₋₃ alkoxy group, a C₁₋₆ alkyl group or a C₂₋₆ alkenyl group, each optionally substituted by a hydroxy or C₁₋₃ alkoxy group; or R¹ and R², are taken together and form a C₃₋₈ cycloalkyl or C₅₋₈ cycloalkenyl group; or both R³, taken together, are a C₁₋₃ alkanediyl group; R⁴ and R⁵, independently from each other, are a hydrogen atom, a methyl or an ethyl group;
the process comprising a hydroformylation and an elimination step starting from a compound of formula (II) 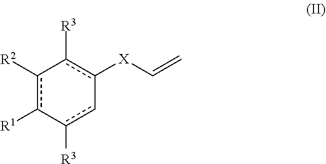
in the form of any one of its stereoisomers or a mixture thereof, and wherein the dotted lines, R¹, R² and R³ have the same meaning as defined for the compound of formula (I) and X represents a CR⁴(OC(═O)R⁶)CHR⁵ or CHR⁴C(OC(═O)R⁶) (R⁵) group wherein R⁴ and R⁵ have the same meaning as defined for the compound of formula (I) and R⁶ is a hydrogen atom, a C₁₋₃ alkoxy group, a phenyl group or C₁₋₃ alkyl group optionally substituted by one to three halogen atoms.

2. The process according to claim 1, wherein R⁵ is a methyl group.

3. The process according to claim 1, wherein R⁴ is a hydrogen atom.

4. The process according to claim 1, wherein R¹, R² and R³, independently from each other, represent a hydrogen atom or a C₁₋₃ alkyl group.

5. The process according to claim 1, wherein the compound of formula (I) is of formula 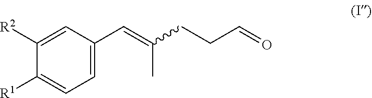
in the form of any one of its stereoisomers or a mixture thereof;
and said compound of formula (II) is of formula 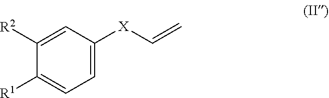
in the form of any one of its stereoisomers or a mixture thereof, and wherein X represents a CH(OC(═O)R⁶)CHMe or CH₂C(OC(═O)R⁶)(Me) wherein R⁶ is a hydrogen atom, a C₁₋₃ alkoxy group, a phenyl group or C₁₋₃ alkyl group optionally substituted by one to three halogen atoms.

6. The process according to claim 1, wherein R⁶ is a methyl group.

7. The process according to claim 1, wherein R² is a hydrogen atom.

8. The process according to claim 1, wherein R¹ is a methyl or an ethyl group.

9. The process according to claim 1, wherein the process comprises the step of
a) hydroformylation of compound of formula (II) to obtain compound of formula 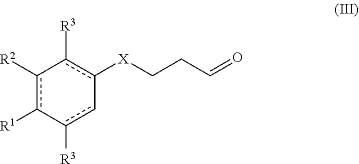
in the form of any one of its stereoisomers or a mixture thereof;
b) protection of the aldehyde group of compound formula (III) obtained in step a) in the form of an acetal of formula 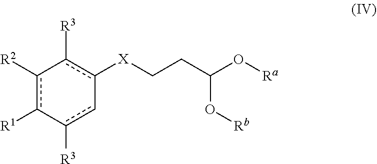
in the form of any one of its stereoisomers or a mixture thereof, wherein R^{a} and R^{b}, independently from each other, represent a C₁₋₄ alkyl group or Rª and R^{b} are taken together and represent a C₂₋₆ alkanediyl group;
c) elimination of the OC(═O) R⁶ group of the compound of formula (IV), optionally followed by an isomerisation to form a compound of formula 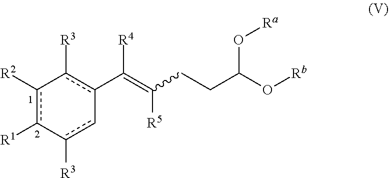
in the form of any one of its stereoisomers or a mixture thereof; and
d) deprotection of the acetal group to obtain compound of formula (I).

10. The process according to claim 1, wherein the hydroformylation is performed in the presence of a rhodium catalyst.

11. A compound of formula 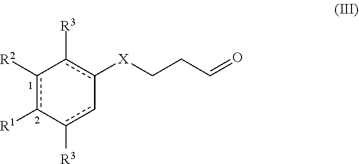
in the form of any one of its stereoisomers or a mixture thereof, and wherein all the dotted lines represent double bonds or the dotted line between carbon 1 and carbon 2 is a carbon-carbon single bond or a carbon-carbon double bond and the other dotted lines are carbon-carbon single bonds; each R¹, R² and R³, independently from each other, represent a hydrogen atom, a C₁₋₃ alkoxy group, a C₁₋₆ alkyl group or a C₂₋₆ alkenyl group, each optionally substituted by a hydroxy or C₁₋₃ alkoxy group; or R¹ and R², are taken together and form a C₃₋₈ cycloalkyl or C₅₋₈ cycloalkenyl group; or both R³, taken together, are a C₁₋₃ alkanediyl group; X represents a CR⁴(OC(═O)R⁶)CHR⁵ or CHR⁴C(OC(═O)R⁶)(R⁵) wherein R⁴ and R⁵, independently from each other, are a hydrogen atom, a methyl or an ethyl group and R⁶ is a hydrogen atom, a C₁₋₃ alkoxy group, a phenyl group or C₁₋₃ alkyl group optionally substituted by one to three halogen atoms, provided that 5-oxo-1-phenylpentyl acetate is excluded.

12. A compound of formula 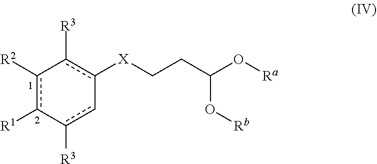
in the form of any one of its stereoisomers or a mixture thereof, and wherein all the dotted lines represent double bonds or the dotted line between carbon 1 and carbon 2 is a carbon-carbon single bond or a carbon-carbon double bond and the other dotted lines are carbon-carbon single bonds; each R¹, R² and R³, independently from each other, represent a hydrogen atom, a C₁₋₃ alkoxy group, a C₁₋₆ alkyl group or a C₂₋₆ alkenyl group, each optionally substituted by a hydroxy or C₁₋₃ alkoxy group; or R¹ and R², are taken together and form a C₃₋₈ cycloalkyl or C₅₋₈ cycloalkenyl group; or both R³, taken together, are a C₁₋₃ alkanediyl group; X represents a CR⁴(OC(═O)R⁶)CHR⁵ or CHR⁴C(OC(═O)R⁶)(R⁵) wherein R⁴ and R⁵, independently from each other, are a hydrogen atom, a methyl or an ethyl group and R⁶ is a hydrogen atom, a C₁₋₃ alkoxy group, a phenyl group or C₁₋₃ alkyl group optionally substituted by one to three halogen atoms; R^{a} and R^{b}, independently from each other, represent a C₁₋₄ alkyl group or R^{a} and R^{b} are taken together and represent a C₂₋₆ alkanediyl group.

13. A compound of formula (V) 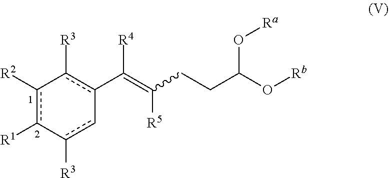
in the form of any one of its stereoisomers or a mixture thereof, and wherein all the dotted lines represent double bonds or the dotted line between carbon 1 and carbon 2 is a carbon-carbon single bond or a carbon-carbon double bond and the other dotted lines are carbon-carbon single bonds; each R¹, R² and R³, independently from each other, represent a hydrogen atom, a C₁₋₃ alkoxy group, a C₁₋₆ alkyl group or a C₂₋₆ alkenyl group, each optionally substituted by a hydroxy or C₁₋₃ alkoxy group; or R¹ and R², are taken together and form a C₃₋₈ cycloalkyl or C₅₋₈ cycloalkenyl group; or both R³, taken together, are a C₁₋₃ alkanediyl group; R⁴ and R⁵, independently from each other, are a hydrogen atom, a methyl or an ethyl group; R^{a} and R^{b}, independently from each other, represent a C₁₋₄ alkyl group or R^{a} and R^{b} are taken together and represent a C₂₋₆ alkanediyl group; provided that 2-(4-cyclohexylbut-3-en-1-yl)-1,3-dioxolane, 2-(4-phenylbut-3-en-1-yl)-1,3-dioxolane and 2-(4-phenylpent-3-en-1-yl)-1,3-dioxolane are excluded.

14. The process according to claim 9, wherein R^{a} and R^{b} are taken together and represent a (CH₂)ₙ group wherein n is 2 or 3.

15. The compound according to claim 12, wherein R^{a} and R^{b} are taken together and represent a (CH₂)ₙ group wherein n is 2 or 3.

16. The compound according to claim 13, wherein R^{a} and R^{b} are taken together and represent a (CH₂)ₙ group wherein n is 2 or 3.
